(19) United States
(12) Patent Application Publication
(10) Pub. No.: US 2014/0350274 A1
(43) Pub. Date: Nov. 27, 2014

(54) METHODS FOR ASYMMETRIC E POXIDATION USING FLOW REACTORS

(71) Applicant: Corning Incorporated, Corning, NY (US)

(72) Inventor: Patrick Rosaire Bazinet, Somerville, MA (US)

(73) Assignee: Corning Incorporated, Corning, NY (US)

(21) Appl. No.: 14/358,798

(22) PCT Filed: Nov. 6, 2012
(86) PCT No.: PCT/US12/63677

§ 371 Date: May 16, 2014

Related U.S. Application Data
(60) Provisional application No. 61/561,023, filed on Nov. 17, 2011.

(51) Int. Cl. C07D 301/12

(52) U.S. Cl. CPC C07D 301/12
USPC 549/523

(57) ABSTRACT

Embodiments of the present disclosure relate to asymmetric epoxidation of olefinic alcohols, using a chiral alcohol chelated titanium catalyst and an organic peroxide performed in a microreactor flow reactor system that can comprise multiple microreactor modules. Molecular sieves can be used to remove any adventitious water in the reagent feed solutions and ensure an anhydrous reaction solution. The use of a microreactor flow reactor allows for the epoxidation reaction to be run at elevated temperatures of at least 20, 30, or even 50° C., which dramatically accelerates the reaction, but without a large drop in enantioselectivity. The reaction can therefore be performed with short reaction times resulting in a high throughput.

### BACKGROUND

This application claims the benefit of priority under 35 U.S.C. §119 of U.S. Provisional Application Ser. No. 61/561,023 filed Nov. 17, 2011 the content of which is relied upon and incorporated herein by reference in its entirety.

### FIELD

The present disclosure generally relates to apparatuses, systems, and methods for asymmetric epoxidation using flow reactors.

### TECHNICAL BACKGROUND

Epoxides are of great importance as reactive intermediates in the synthesis of many compounds used in the pharmaceutical and fine chemical industries, for example, cosmetics and polymer industries. Enantioenriched chiral epoxides are particularly valuable since they provide access to a multitude of other chiral compounds, such as, but not limited to chiral alcohols, diols, amino alcohols, polyethers, due to the propensity of epoxides to participate in ring-opening reactions. One reliable way of preparing chiral epoxides is via asymmetric epoxidation of olefins. Established methods for asymmetric epoxidation of olefins include the titanium-tartrate catalyzed epoxidation of allylic alcohols, manganese-salen catalyzed epoxidation of olefins, and fructose-derived ketone catalyzed Oxone oxidation of olefins.

Although chiral glycidol derivatives can be prepared via asymmetric epoxidation of allylic alcohols, performing the reaction at a useful scale can be challenging. The reaction is usually executed in multiple phases comprising of in-situ formation of the catalyst followed by controlled addition of reagents. The presence of water and variations in reaction temperature can lead to catalyst decomposition and loss of selectivity. The reactions are typically run at low temperatures, usually between −50° C. and 10° C., depending on the step involved, and for extended periods of time, usually multiple hours total. Due to these factors, the costs associated with the synthesis of chiral epoxides can be prohibitive. There is a need for more efficient methods of preparing chiral epoxides.

### SUMMARY

Microreactors, microfluidic devices possessing channels ranging from microns to millimeters, have been designed and used to perform many chemical transformations. The extremely high surface area to volume ratios, high heat transfer, and reduced process volumes associated with microreactors makes them particularly suitable “process intensification.” Microreactors can be used to assemble flow systems that maximize mass- and heat-transfer and therefore lead to major improvements in the manufacturing of compounds through a decrease in equipment size, energy consumption and waste production all while increasing production capacity. Microreactors are also well suited to continuous manufacturing of compounds which can lead to higher product quality and lower production costs over traditional batch synthesis. Microreactor technology may be used to develop an “intensified” process for performing the asymmetric epoxidation of allylic alcohols.

Embodiments of the present disclosure relate to asymmetric epoxidation of olefinic alcohols, using a chiral-alcohol chelated metal catalyst and an organic peroxide, performed in a flow system preferably comprised of multiple microreactor modules, with in-situ generation of the catalyst followed by epoxidation then quenching, with temperature of the oxidation step (and optionally of all three steps of catalyst generation, oxidation, and quenching) being at least 20° C., at least 30° C., or at least 50° C., simultaneously with enantioselectivity of at least 85% or at least 88 or 90%, and epoxide yield of at least 82%, desirably at least 90%, more desirably even at least 94%, desirably with epoxidation time of 4 minutes or less, desirably 2 minutes or less or even 1 minute or less. The chiral alcohol is desirably (+)-Diethyl L-tartrate ((+)-DET). The metal catalyst is desirably Titanium (oxide) from an alkoxide precursor, preferably Ti(OiPr)₄ (titanium (tetra)isopropoxide) as precursor, but may generally be a metal of Groups 4b to 6b of atomic number 22 to 74, particularly those metals of groups 4b to 5b of atomic number 22 to 73 of the Periodic Chart, from an appropriate alkoxide precursor. Particular metals of interest include, tantalum, zirconium, hafnium, niobium, vanadium, and molybdenum, in addition to the preferred titanium. Reactants are carried in an inert organic solvent. Halogenated hydrocarbons such as methylene chloride, dichloroethane, carbon tetrachloride, are desirable, with methylene chloride presently believed to be most desirable. The reaction should be performed in the absence of water. Inline packed-beds of molecular sieves are desirably used to remove any adventitious water in the reagent feed solutions and ensure an anhydrous reaction.

The use of a thermally controlled microreactor flow system with good thermal control and relatively fast heat and mass transfer allows for the epoxidation reaction to be run at elevated temperatures which dramatically accelerates the reaction, but without too a significant drop in enantioselectivity. The short residence times thus achievable result in high throughput. The use of higher temperatures also eliminates the need to use cryogenic conditions, reducing cost.

Desirably, the steps disclosed method(s) are performed in multiple fluidic modules, fluidically connected in series. Desirably, one (or more) modules is used for each of the main steps (generation of catalyst, epoxidation, quenching). Performing the reaction under continuous-flow conditions using multiple microreactor modules allows for easy optimization of the three reaction steps by performing each step in one (or more) modules well-suited to the respective step. Using such a continuous flow system with the resulting performance achievable decreases labor requirements, minimizes process volume and safety concerns, and permits continuous manufacturing of the compound, relative to competing batch techniques. With the tight thermal and process control provided in the micro fluidic flow reactor, higher temperatures may be employed for epoxidation than are normally achievable, without too severe a reduction in enantioselectivity. The high temperatures allows for high yield of epoxides in short reaction times, boosting production rates. The use of a flow system also offers the possibility of easily increasing the production scale by simply “numbering-up” the number of systems. Use of Corning's Advanced Flow Reactor modules allows for potential scale-up from the low-flow modules used experimentally herein, through the G1, G2, G3 and G4 modules for a 300-fold or greater increase in production, under sufficiently similar fluid- and thermo-dynamic conditions to maintain the productivity advantages of the disclosed methods, before (external) parallelization of the reactor would be required.

Additional features and advantages will be set forth in the detailed description which follows, and in part will be readily apparent to those skilled in the art from that description or recognized by practicing the embodiments as described herein, including the detailed description which follows, the claims, as well as the appended drawings.

It is to be understood that both the foregoing general description and the following detailed description are merely exemplary, and are intended to provide an overview or framework to understanding the nature and character of the claims. The accompanying drawings are included to provide a further understanding, and are incorporated in and constitute a part of this specification. The drawings illustrate embodiments, and together with the description serve to explain principles and operation of the various embodiments.

### BRIEF DESCRIPTION OF THE DRAWINGS

The components of the following figures are illustrated to emphasize the general principles of the present disclosure and are not necessarily drawn to scale. The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 schematically depicts the flow system used to perform asymmetric epoxidation according to one or more embodiments described and illustrated herein;

FIG. 2 is a plot of the kinetic profile of asymmetric epoxidation reaction at different temperatures according to one or more embodiments described and illustrated herein;

FIG. 3A is a plot of the effect of reaction temperature on the enantioselectivity according to one or more embodiments described and illustrated herein; and

FIG. 3B is a plot an expanded view of FIG. 3A regarding the effect of reaction temperature on the enantioselectivity according to one or more embodiments described and illustrated herein.

### DETAILED DESCRIPTION

Some aspects of the present disclosure are directed to asymmetric epoxidation performed in a flow system comprising of multiple microreactor plates. According to various embodiments, the asymmetric epoxidation flow system, as well as associated methods of using the asymmetric epoxidation flow system, described herein develops an intensified process for performing the asymmetrical epoxidation of epoxides. The asymmetric epoxidation flow system comprising of multiple microreactor plates will be described in further detail herein with specific reference to the figures and conducted experiments.

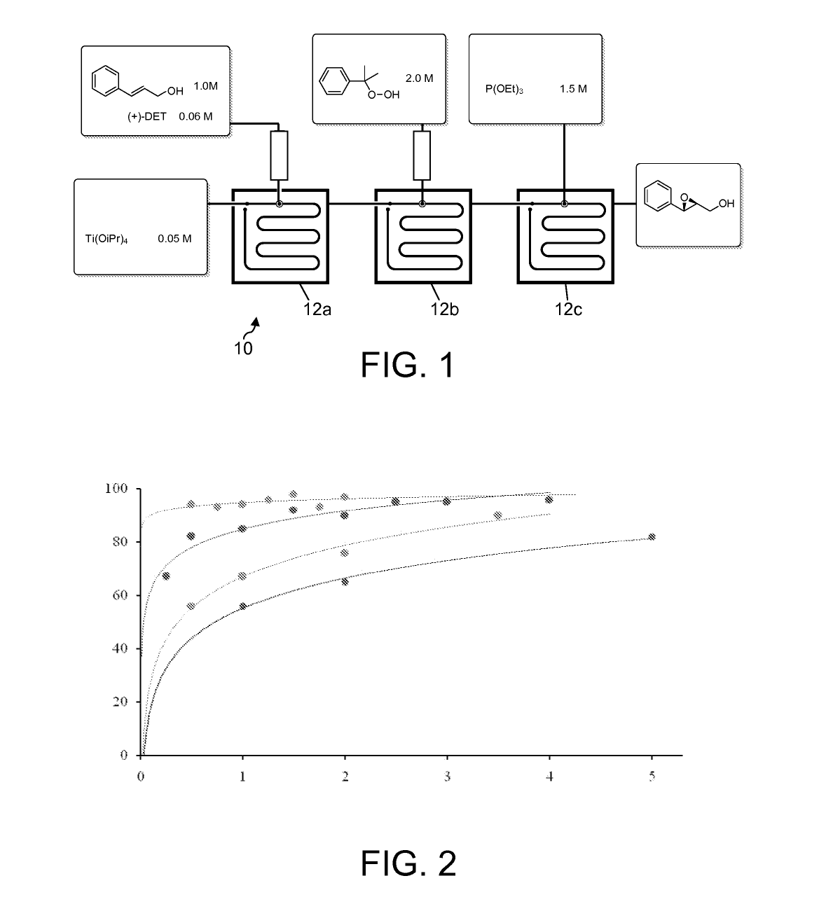

FIG. 1 schematically represents the flow system 10 used to perform the experimental reaction of asymmetric epoxidation of cinnamyl alcohol. The feed solutions are desirably prepared by dissolving the reagents an inert organic solvent. Methylene chloride is presently preferred and was used experimentally, in amounts sufficient to provide solutions of the desired concentrations, as indicated in FIG. 1. The feed solutions are pumped into the flow system 10 at the appropriate flow rates, given below for multiple experiments in Table 1. The temperature of the flow system 10 is controlled by a heat exchange fluid that is circulated and regulated by a circulating heater/chiller bath, not shown. The reaction occurs in three distinct phases, each performed in one of the three modules 12a, 12b, 12c, which are fluidically connected in series (left-to-right in the figure).The catalyst is generated in the first module 12a, epoxidation occurs in the second module 12b, and quenching of excess peroxide happens in the third module 12c.

FIG. 2 is a plot showing the percentage yield of the desired product, 3-phenyl-glycidol, on the y-axis, measured when the reaction was performed at various temperatures and reaction/residence times. The four experimental temperatures correspond to the four traces, namely 10°, 20°, 30° and 50° C., from lowest to highest trace. The total epoxidation reaction time (corresponding to the residence time of the reagents in the module 12b) is given in minutes on the x-axis, with the reaction was performed using the flow system according to FIG. 1.

Table 1 illustrates the details of data shown in FIG. 2 for experiments conducted using the flow system according to FIG. 1 during the various temperature and reaction/resident time of the flow rate of the solution and the corresponding epoxide yield percentage, with the doubled lined boxes indicating performance within desirable ranges.

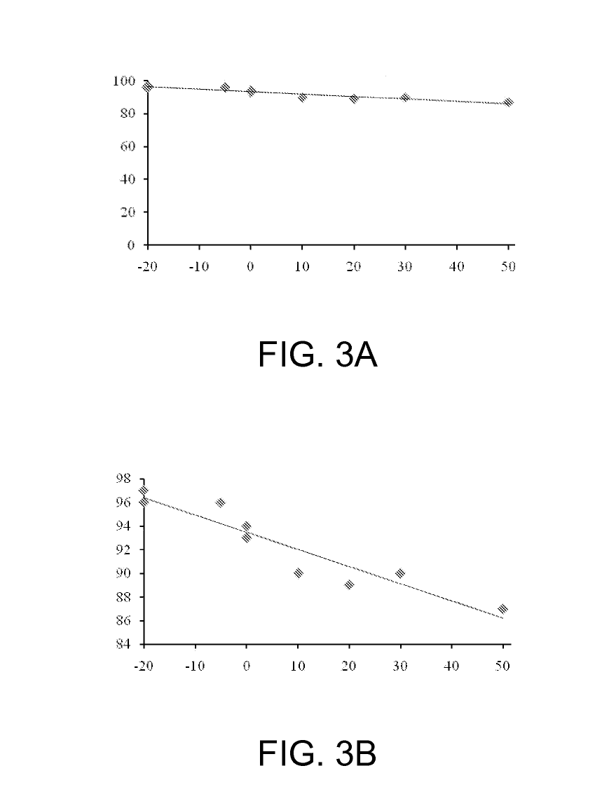

FIG. 3A is a plot showing the value of the enantiomeric excess of the product, 3-phenyl-glycidol, as a percentage on the y-axis, obtained when the epoxidation reaction was performed at various temperatures, labeled in degrees C. on the x-axis, with the reaction again performed using the flow system according to FIG. 1. The temperature value represents the temperature of all three modules, 12a, 12b, 12c of the flow system, since they were all connected in series in a heat exchange loop (not shown in FIG. 1).

FIG. 3B is a plot showing the data of FIG. 3A, but with an expanded view of the y-axis.

TABLE 1

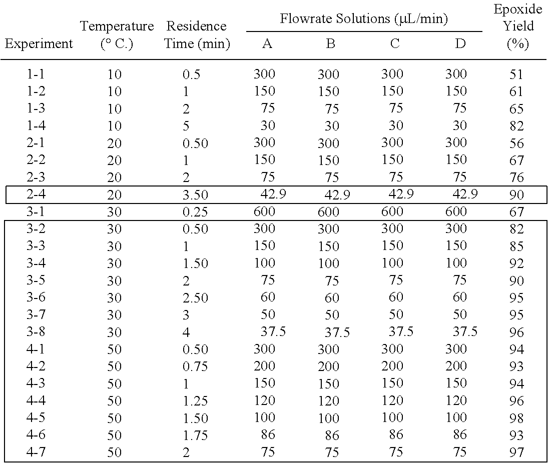

Asymmetric epoxidation is generally considered to be rather sensitive to temperature, but as FIG. 3 clearly shows, performing the reaction at elevated temperatures results in only a minor loss in enantioselectivity in the temperature-controlled microfluidic flow reactor environment. The selectivity was highest for reactions performed at −20° C. at 96%, but decreased only slightly to 87% when run at 50° C. This relatively slight loss in selectivity is more than compensated for by the drastic increase in reaction rate and concomitant increase in throughput achievable at 20°, 30° and particularly at 50° C.

Using the flow system according to FIG. 1, from the date in Table I and FIG. 3A, it may be seen that the throughput in terms of amount of epoxide enantiomer produced when operating at 50° C. and 30 sec residence time (94%) would be 2.5 g/h or 61 g/day, easily achieving a target of 50 g/day. If the system was expanded to comprise a total of nine microreactor modules (three series of three in parallel), the throughput of the system would be tripled to 7.6 g/h. In this situation, the flow rate for each reagent stream would be approximately 0.9 mL/min. A further increase in throughput could be achieved by utilizing the Corning G1 Flow Reactor. With the G1, with an average volume of 8 mL, a single reactor of three modules in series could produce over 1 kg per day. If a total of nine modules were used (three series of three arranged in parallel), the production rate of epoxide enantiomer would be 135 g/h or 3.2 kg/day.

It is noted that terms like “typically,” or “preferably” when utilized herein, are not intended to limit the scope of the disclosure or to imply that certain features are critical, essential, or even important to the structure or function of the claimed invention. Rather, these terms are merely intended to highlight alternative or additional features that may or may not be utilized in a particular embodiment of the present disclosure.

For the purposes of describing and defining the present disclosure it is noted that the terms “approximately” and “about” are utilized herein to represent the inherent degree of uncertainty that may be attributed to any quantitative comparison, value, measurement, or other representation.

1. A method of performing asymmetric epoxidation of olefinic alcohols, the method comprising:
providing a flow reactor including thermal control having channels with a sub-millimeter to multiple-millimeter scale cross section;
generating a chiral-alcohol chelated transition metal catalyst within the flow reactor and mixing the chiral-alcohol chelated transition metal catalyst with an olefinic alcohol within the flow reactor, forming a first mixture comprising the chiral-alcohol chelated transition metal catalyst and the olefinic alcohol;
epoxidizing the olefinic alchohol by flowing a stoichometerically excess amount of an organic hydroperoxide into the flow reactor and mixing the organic hydroperoxide with the first mixture, forming a second mixture comprising the chiral-alcohol chelated transition metal catalyst, the partly or wholly epoxidized olefinic alcohol and the remaining organic hydroperoxide; and
quenching the second mixture by flowing a reducing agent into the flow reactor and mixing the reducing agent with the second mixture,
wherein the epoxidation step is performed within a temperature-controlled portion of the flow reactor at a temperature of at least 20° C. and within a time of 4 minutes or less, with an epoxide yield of at least 82% and an enantioselectivity of at least 85%.

2. The method according to claim 1 wherein the step of providing a flow reactor comprises providing at least three flow reactor modules fluidically connected in series, with a first one or more flow reactor modules of the at least three modules used for the step of generating, a second one or more of the at least three modules used for the step of epoxidizing, and a third one or more flow reactor modules of the at least three modules used for the step of quenching.

3. The method according to claim 1 wherein the epoxidation step is performed within a temperature-controlled portion of the flow reactor at a temperature of at least 30° C.

4. The method according to claim 1 wherein the epoxidation step is performed within a temperature-controlled portion of the flow reactor at a temperature of at least 50° C.

5. The method according to claim 3 wherein the epoxidation step is performed within a time of 2 minutes or less.

6. The method according to claim 3 wherein the epoxidation step is performed within a time of 1 minute or less.

7. The method according to claim 1 wherein the step of generating comprises generating a metal catalyst in which the metal is one or more of tantalum, zirconium, hafnium, niobium, vanadium, and molybdenum, and titanium.

8. The method according to claim 7 wherein the step of generating comprises generating a metal catalyst in which the metal is titanium.

9. The method according to claim 1 wherein the step of epoxidizing by flowing a stoichometerically excess amount of an organic hydroperoxide comprises flowing cumene hydroperoxide.

10. The method according to claim 1 wherein the steps of generating, epoxidizing and quenching each comprise flowing reactants in an inert organic solvent.

11. The method according to claim 10 wherein flowing reactants in an inert organic solvent comprises flowing reactants in methylene chloride.

12. The method according to claim 1 further comprising the step of using a molecular sieve material to remove water from one or more reactant streams entering the flow reactor.

13. The method according to claim 1 wherein the step of generating a chiral-alcohol chelated transition metal catalyst within the flow reactor comprises mixing a transition-metal catalyst precursor with (+)-Diethyl L-tartrate.

14. The method according to claim 1 wherein the process has a production rate of a desired enantiomer of a desired epoxide at least 50 g per day per series-connected flow reactor.

15. The method according to claim 1 wherein the process has a production rate of a desired enantiomer of a desired epoxide at least 1000 g per day per series-connected flow reactor.
